(19) United States
(12) Patent Application Publication
(10) Pub. No.: US 2024/0321415 A1
(43) Pub. Date: Sep. 26, 2024

(54) MEDICAL IMAGE DISPLAY APPARATUS, MEDICAL IMAGE DISPLAY SYSTEM, MEDICAL IMAGE DISPLAY METHOD, AND COMPUTER-READABLE RECORDING MEDIUM STORING PROGRAM

(71) Applicant: KONICA MINOLTA, INC., Tokyo (JP)

(72) Inventors: Hidekazu TAKAHASHI, Osaka (JP); Hisashi WADA, Tokyo (JP); Ken SATOU, Tokyo (JP); Ryuta OGAWA, Osaka (JP); Seiji NOMURA, Tokyo (JP); Kohei IKEDA, Tokyo (JP)

(73) Assignee: KONICA MINOLTA, INC., Tokyo (JP)

(21) Appl. No.: 18/583,912

(22) Filed: Feb. 22, 2024

(30) Foreign Application Priority Data
Mar. 24, 2023 (JP) 2023-048398

(51) Int. Cl. G16H 10/60; G06F 3/147

(52) U.S. Cl. CPC G16H 10/60; G06F 3/147

(57) ABSTRACT

Disclosed is a medical image display apparatus including: a display part that displays a medical image of a target first patient; and a communicator that communicates with a patient database storing person identifying information on patients generated in a plurality of medical facilities. The display part displays existence information when a second patient is recorded in the patient database, the second patient being a related patient that has person identifying information similar to person identifying information on the first patient.

### BACKGROUND OF THE INVENTION

### Technical Field

The present invention relates to a medical image display apparatus, a medical image display system, a medical image display method, and a computer-readable recording medium storing a program.

### Description of Related Art

In recent years, medical facilities such as hospitals and clinics have been increasingly segmetalized in terms of function. In addition, the treatment (care cycle: inspection, diagnosis, treatment, and the like) for one disease of a patient has been spread over a plurality of medical facilities. In addition, with the development of a cloud system, a technology for managing data of a plurality of medical facilities in a cloud has been developed. However, under the present circumstances, identification information (patient ID or the like) for identifying a patient varies among medical facilities. Therefore, it is difficult to comprehensively grasp data on a patient existing over a plurality of medical facilities. Therefore, there is a trend to manage patients with common identification information. The common identification information is, for example, a region ID unique within a region, My Number (R), a medical ID, or the like.

However, it cannot be said that such common identification information is generally used. In each medical facility, it is still common to manage patients by their own identification information (patient ID or the like).

Japanese Unexamined Patent Publication No. 2017-215883 describes a medical image display apparatus that identifies a patient by using a so-called name identification technique. The name identification technique is a technique that includes searching for related patients similar to a search target patient and causing a display means to display patient information on each of the search target patient and the related patients in a list format so that differences between the patient information can be compared. The medical image display apparatus of Japanese Unexamined Patent Publication No. 2017-215883 has a configuration of displaying a name identification list and specifying the same patient in advance by using the name identification list.

### SUMMARY OF THE INVENTION

A user such as a doctor first refers to a medical image of a patient captured at his/her own facility for interpretation. In the invention of Japanese Unexamined Patent Publication No. 2017-215883, a work list for name identification is separately activated at the time of interpretation of a target medical image, and a name identification list for a related patient is searched. If no related patient is present, work efficiency is lowered by the activation of the worklist and the check of the name identification list.

The present invention has been made in consideration of the above-described circumstances. It is an object of the present invention to provide a medical image display apparatus, a medical image display system, a medical image display method, and a computer-readable recording medium storing a program, which are capable of searching for related patient without reducing user's work efficiency.

To achieve at least one of the abovementioned objects, according to an aspect of the present invention, a medical image display apparatus reflecting one aspect of the present invention includes:
a display part that displays a medical image of a target first patient; and
a communicator that communicates with a patient database storing person identifying information on patients generated in a plurality of medical facilities; wherein
the display part displays existence information when a second patient is recorded in the patient database, the second patient being a related patient that has person identifying information similar to person identifying information on the first patient.

To achieve at least one of the abovementioned objects, according to another aspect of the present invention, a medical image display method reflecting one aspect of the present invention includes:
displaying a medical image of a target first patient; and
communicating with a patient database storing person identifying information of patients generated in a plurality of medical facilities, wherein
the displaying includes displaying existence information when a related patient having person identifying information similar to person identifying information of the first patient is recorded in the patient database.

To achieve at least one of the abovementioned objects, according to yet another aspect of the present invention, a computer-readable recording medium storing a program that causes a computer of a medical image display apparatus to function as:
a display controller that causes a display part to display a medical image of a target first patient; and
a communicator that communicates with a patient database storing person identifying information on patients generated in a plurality of medical facilities, wherein
the display controller causes the display part to display existence information when a related patient having person identifying information similar to person identifying information of the first patient is recorded in the patient database.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a block diagram illustrating the configuration of a medical image display system;

FIG. 2 is a block diagram illustrating a configuration of a medical image display apparatus;

FIG. 3 is a flowchart illustrating medical image display processing executed by the medical image display apparatus;

FIG. 4 is a diagram illustrating a work list in which a list of examinations satisfying a predetermined search condition is displayed;

FIG. 5 is a diagram illustrating a viewer when no related patient is present;

FIG. 6 is a diagram illustrating a viewer when a related patient is present;

FIG. 7 is a diagram illustrating a work list after a user operation for existence information;

FIG. 8 is a diagram illustrating an examination list of a related patient;

FIG. 9 is a diagram illustrating a viewer when medical images obtained by examinations in different medical facilities are displayed; and

FIG. 10 is a diagram illustrating another example of the viewer when no related patient is present.

### DETAILED DESCRIPTION

Hereinafter, an embodiment of the present invention will be described below. It should be noted that the claims of the present invention are not limited to the illustrated examples.

### [Medical Image Display System]

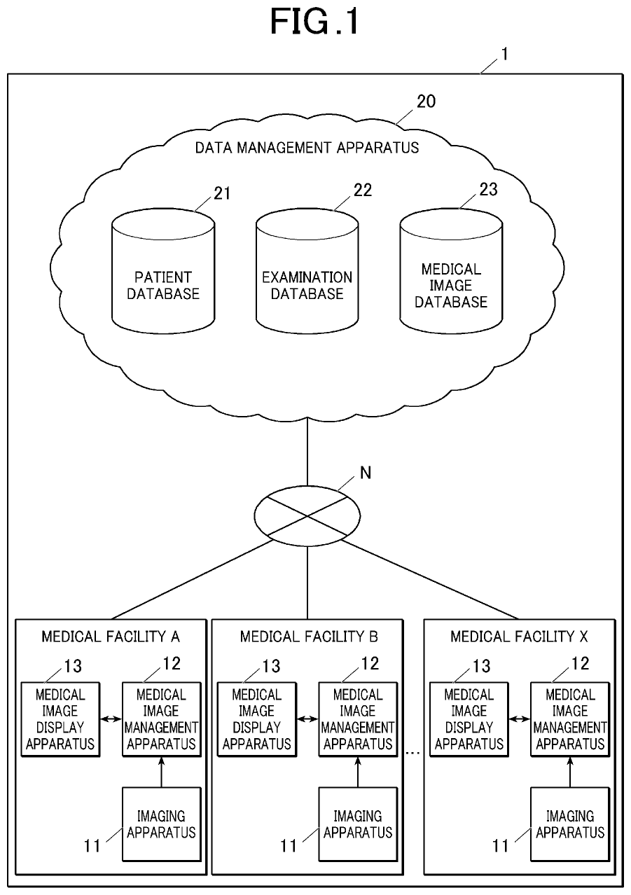

FIG. 1 shows a configuration of a medical image display system 1. The medical image display system 1 includes an imaging apparatus 11, a medical image management apparatus 12 and a medical image display apparatus 13 of each of a plurality of medical facilities A, B . . . X. The medical image display system 1 includes a data management apparatus 20. The apparatuses included in the medical image display system 1 are connected to each other via a communication network N such as a local area network (LAN), a wide area network (WAN), or the Internet. The apparatuses included in the medical image display system 1 conform to the HL7 (Health Level Seven) or DICOM (Digital Image and Communications in Medicine) standard. Therefore, the communication between the apparatuses conforms to HL7 or DICOM.

### [Imaging Apparatus]

The imaging apparatus 11 is, for example, an image generating apparatus such as an X-ray imaging apparatus (DR or CR), an ultrasound diagnostic apparatus (US), a CT, or an MRI. The imaging apparatus 11 captures an image of an examination target part of a patient as a subject and generates a medical image as medical information. The generation of a medical image by the imaging apparatus 11 is based on examination order information transmitted from a radiology information system (RIS) (not illustrated) or the like.

Supplementary information (patient information, examination information, an image ID, and the like) is written on the medical image. After the attachment of the supplementary information, the medical image is transmitted to the medical image management apparatus 12 via a console or the like (not illustrated). The supplementary information is written in the header of the image file in accordance with, for example, the DICOM standard. Furthermore, the imaging apparatus 11 may be configured to transmit a medical image from the console to the image analyzing apparatus, and the image analyzing apparatus transmits the analyzed image on which predetermined analysis processing has been performed to the medical image management apparatus 12.

### [Medical Image Management Apparatus]

The medical image management apparatus 12 is, for example, a server constituting a picture archiving and communication system (PACS). The medical image management apparatus 12 associates the medical image received from the imaging apparatus 11 with patient information, examination information, the image ID of the medical image, the facility ID of the medical facility, and the like. The medical image management apparatus 12 stores the medical images in a database. Then, the medical image management apparatus 12 transmits a copy of the medical image stored in the database to the data management apparatus 20.

### [Medical Image Display Apparatus]

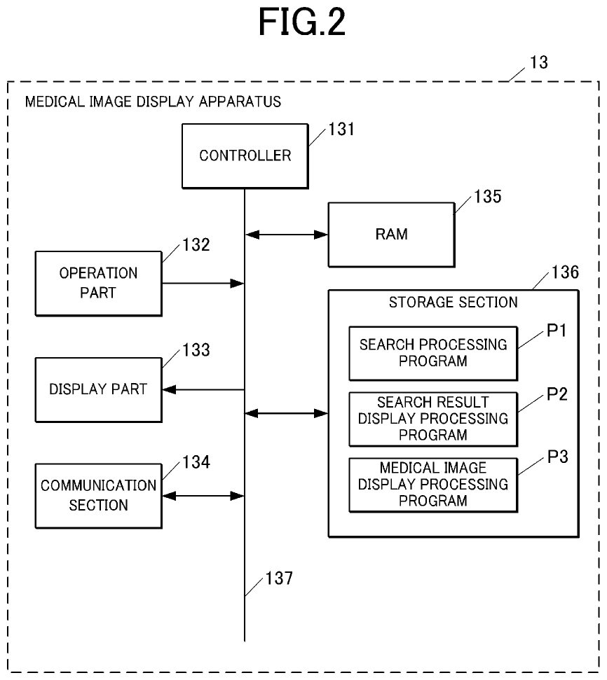

The medical image display apparatus 13 is a device that displays a medical image captured in a specified examination. FIG. 2 is a block diagram illustrating the functional configuration of the medical image display apparatus 13. The medical image display apparatus 13 includes a controller 131, an operation part 132, a display part 133, a communication section (communicator) 134, a RAM (Random Access Memory) 135, and a storage section 136. The parts or sections of the medical image display apparatus 13 are connected to each other by a bus 137.

### (Controller)

The controller 131 is configured by a central processing unit (CPU) or the like. The controller 131 reads a system program and various processing programs stored in the storage section 136. The controller 131 develops the read program in a work area formed in the RAM 135. The controller 131 controls each section or part in accordance with the program developed in the work area.

### (Operation Part)

The operation part 132 includes a keyboard having cursor keys, character input keys, various function keys, and the like, and a pointing device such as a mouse. The operation part 132 outputs, to the controller 131, an instruction signal corresponding to an input by a key operation on the keyboard or a mouse operation. The operation part 132 may include a touch screen formed to cover the display part 133.

### (Display Part)

The display part 133 is a monitor such as a liquid crystal display (LCD). The display part 133 displays various screens and the like in accordance with an instruction which is a display signal input from the controller 131.

### (Communication Section)

The communication section 134 includes a LAN adapter, a router, and a terminal adapter (TA). The communication section 134 transmits and receives data to and from an external device connected to the communication network.

### (RAM)

The RAM 135 forms a work area. In the work area, various kinds of data is temporarily stored in various kinds of processing controlled by the controller 131. The data is, for example, various programs read from the storage section 136, input or output data, parameters, and the like.

### (Storage Section)

The storage section 136 is a hard disk, a semiconductor nonvolatile memory, or the like. The storage section 136 stores a system program and various processing programs to be executed by the controller 131, and data required for executing these programs. The storage section 136 stores, for example, a search processing program P1, a search result display processing program P2, and a medical image display processing program P3.

### {Search Processing Program}

The search processing program P1 is a program for executing examination search processing and related-patient search processing. The examination search processing is processing of searching the medical image management apparatus 12 for an examination that matches a predetermined search condition from and extracting it. The related-patient search processing is processing of searching the patient database 21 for a patient and extracting it. The patient to be searched for in the related-patient search processing is the second patient having the person identifying information that matches the person identification information on the first patient.

### {Search Result Display Processing Program}

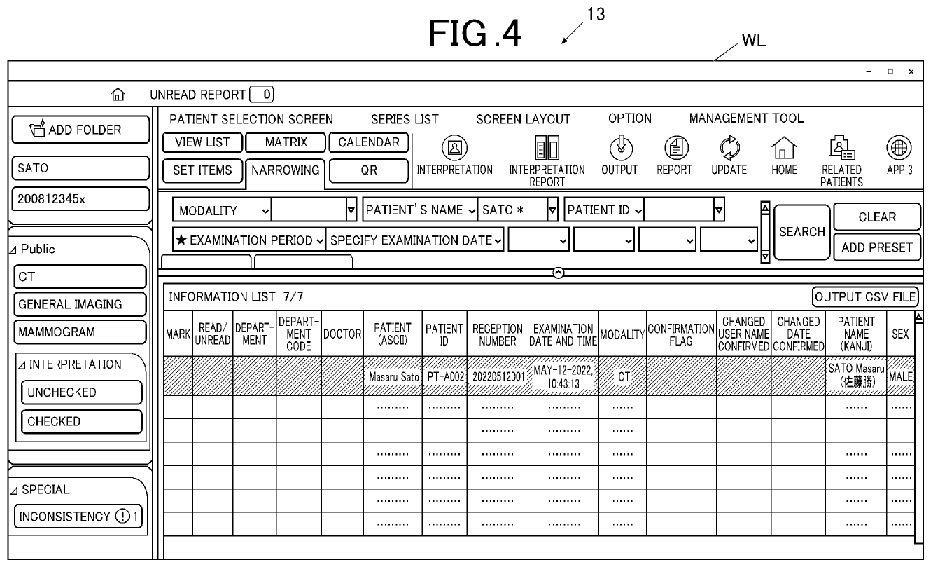

The search result display processing program P2 is a program for executing related-patient search result display processing and examination search result display processing. The examination search result display processing is processing of displaying, in the work list WL (see FIG. 4), a list of examinations extracted by the search processing program P1. The related-patient search result display processing is processing of displaying, in the work list WL, a list of related patients extracted by the search processing program P1.

### {Medical Image Display Processing Program}

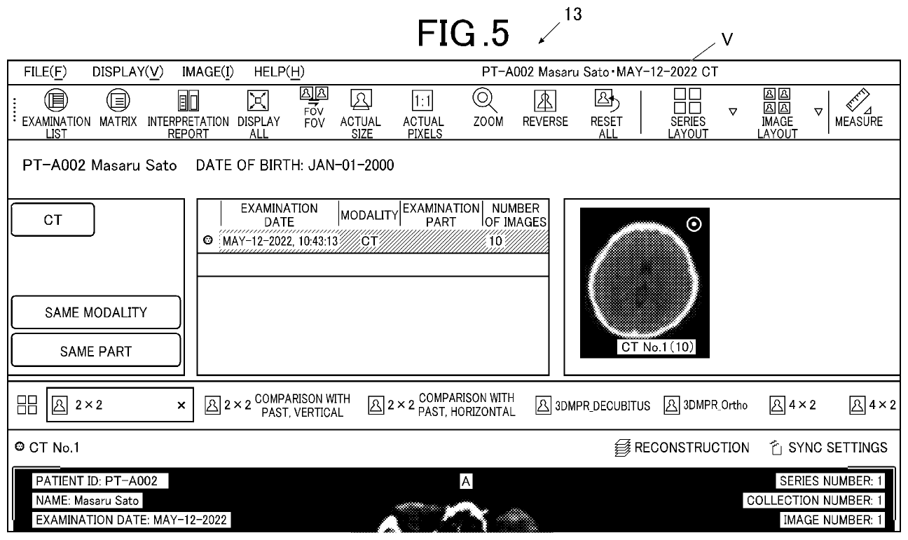

The medical image display processing program P3 is a program for executing medical image display processing. The medical image display processing starts with receiving designation of one examination from the list of examinations displayed in the examination search result display processing. The medical image display processing then acquires a medical image of the designated examination from the medical image management apparatus 12 or the medical image database 23 and displays the medical image on the viewer V (see FIG. 5).

### [Data Management Apparatus]

Returning to FIG. 1, the data management apparatus 20 is, for example, a virtual device provided in a cloud environment. The data management apparatus 20 is network-connected to the medical image management apparatuses 12 and the medical image display apparatuses 13 in a plurality of medical facilities. The data management apparatus 20 includes a patient database 21, an examination database 22, a medical image database 23, and the like, which corresponds to each medical facility. The data management apparatus 20 stores a medical image received from the medical image management apparatus 12 of each medical facility to the medical image database 23 corresponding to the medical facility. The data management apparatus 20 transmits data in the database of each medical facility to the medical image display apparatus 13. The data management apparatus 20 transmits the data in the database of each medical facility in response to communication from the medical image display device 13 of each medical facility.

### {Patient Database}

The patient database 21 stores person identifying information on patients in a plurality of medical facilities. The person identifying information includes a plurality of items (elements). The person identifying information includes, for example, patient information such as patient ID, patient's name (Japanese kanji, Japanese kana, alphabet), date of birth (age), sex, and medical facility.

In the patient database 21, each of patient information may be associated with medical information such as medical record information and vital information. The medical information is generated at the same medical facility as that of the patient information associated with the medical information.

### {Examination Database}

The examination database 22 stores examination identifying information on examinations performed at a plurality of medical facilities. The examination identifying information includes a plurality of items (elements). The examination identifying information includes, for example, an examination ID, a patient name (Japanese kanji, Japanese kana, and alphabets), a date of birth, a sex, a medical facility, an examination date, a modality, and an imaging part. The items of examination identifying information include a path of an medical image of the patient captured in the examination and saved in the medical image database 23.

### {Medical Image Database}

The medical image database 23 stores medical images captured by the image capturing apparatus 11 in examinations performed in a plurality of medical facilities and received from the medical image management apparatus 12. The medical image database 23 also stores supplementary information in association with each medical image. The supplementary information includes patient information, examination information, series information, image information, and the like.

The patient information is information on a patient. The patient information includes a patient ID, a patient name, a date of birth, an age, a sex, and the like.

The examination information is information on an examination. The examination information includes an examination instance UID, an examination date and time, and an examination description.

The series information is information on a series. The series information includes a modality (CT, MRI, DR, CR, US, or the like), a series instance UID, an examination part, a series number, a series description, and the like. The series instance UID is a unique ID for identifying the series. The series number is a number indicating the order of the series in the same examination. The series number may be used for determining a display order or the like.

The image information is information on a medical image. The image information includes an image number, an image generation date and time, an imaging direction, an imaging start position, an image type, a file path, and the like. The image number is a number indicating the ordinal number of an image in the same series. The imaging start position indicates coordinates at which the examination (scan) is started in an examination such as CT or MRI. The image type is information indicating whether the image is a slice image or a scout image in an examination such as CT or MRI. The file path is information indicating a storage location of the medical image in the medical image database 23.

### [Medical Image Display Processing]

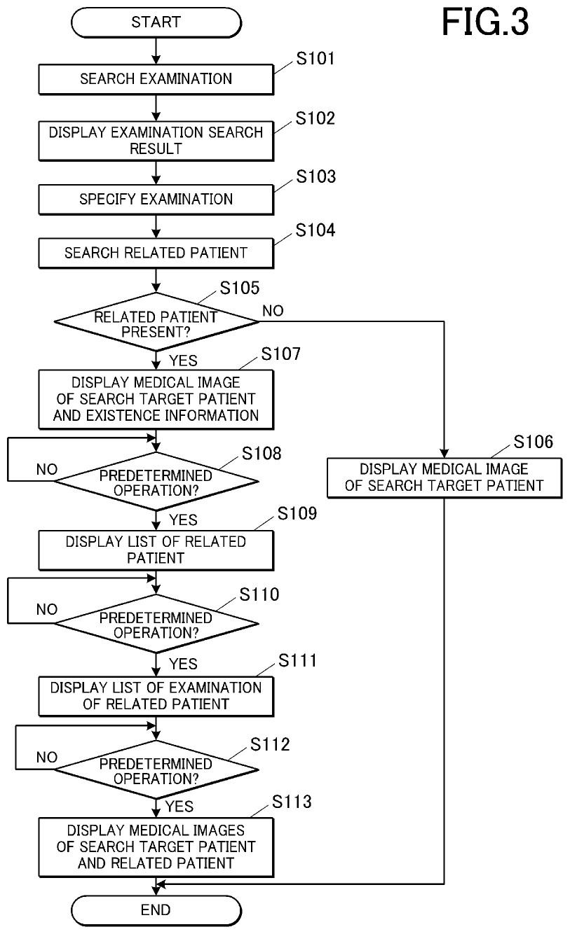

Next, the operation of the medical image display apparatus 13 will be described. FIG. 3 is a flowchart illustrating medical image display processing executed by the medical image display apparatus 13. The medical image display processing is implemented by software processing or the like in cooperation with the controller 131 and a program stored in the storage section 136. The medical image display processing starts with activation of the work list WL.

The user inputs predetermined search conditions by operating the operation part 132. After inputting the search conditions, the user operates a search button. The controller 131 executes the examination search processing with the search processing program P1 (step S101). The controller 131 acquires the examination data of its medical facility satisfying a predetermined search condition from the medical image management apparatus 12.

The controller 131 executes the examination search result display processing with the search result display processing program P2 (step S102). Through the examination search result display processing, the controller 131 displays a list of inspections satisfying predetermined search conditions in the work list WL. FIG. 4 illustrates an example of the work list WL in which a list of examinations satisfying a predetermined search condition is displayed. In FIG. 4, as the predetermined search condition, a list of examinations performed for a patient with a name starting with “ [symbol] (SATO)” is displayed in the work list WL.

The controller 131 receives a predetermined user operation to designate the first examination (step S103). For example, in FIG. 4, the user clicks the row of “ [symbol] (SATO Masaru)” with the patient ID of PT-A002 once to select it. Then, the user double-clicks the selected row to designate the examination of the row. The controller 131 acquires the medical image of the first examination specified in this step from the medical image management apparatus 12.

Back to FIG. 3, the controller 131 that has received the designation of the first examination executes the related-patient search processing with the search processing program P1 (step S104). Then the controller 131 acquires, from the patient database 21, data on a related patient (second patient) having person identifying information similar to that of the first patient in the specified first examination.

In the present embodiment, the controller 131 executes the related-patient search processing by use of so-called name identification search. The name identification search is a search method of searching for, based on first person identifying information identifying a first patient, a second patient (related patient) having person identifying information (second person identifying information) similar to the first person identifying information.

The related-patient search processing based on the name identification search by the controller 131 will be described. The name identification search includes, for example, using the patient information of the first patient stored in the patient database 21 of the first medical facility for searching for a related patient stored in the patient database 21 of the first medical facility. The name identification search includes using the patient information of the first patient stored in the patient database 21 of the first medical facility for searching for a related patient stored in the patient database 21 of the second medical facility. It is needless to say that the related patient to be searched by the name identification search may be the same person as the search target patient.

In the name identification search, for example, five items, i.e. the name (Japanese kanji, Japanese kana, and alphabet), the date of birth, and the sex of the patient among the person identifying information are used as the name identification conditions. A search condition as to which item of the person identifying information is used as the name identification conditions is stored in the storage section 136 in advance.

The controller 131 compares the person identifying information between the search target patient and other patients with respect to each item. Then, the controller 131 adds the penalty value of the mismatched item to the reference value “1”, and calculates the reciprocal thereof as the similarity between the patients.

For example, it is assumed that the patients have the same name (Japanese kana), the same name (alphabet), and the same sex. Further, it is assumed that the patients do not have the same name (Japanese kanji) and the same date of birth. The penalty value is assumed to be set as follows, for example.
Name (Japanese kanji): 5
Name (Japanese kana): 1
Name (alphabetic): 1
Date of birth: 3
Sex: 1

In this case, the similarity is obtained as follows.

$$Similarity = 1 / \{ Reference value + Penalty value of name ( Japanese kanji ) + penalty value of date of birth \}$$

$$= 1 / 1 + 5 + 3 )$$

$$= 0.11$$

For another example, it is assumed that the patients have the same name (Japanese kanji), date of birth, and sex. Further, it is assumed that patients do not have the same name (Japanese kana) and the same name (alphabet). The penalty value is assumed to be set in the same manner as described above. In this case, the similarity is obtained as follows.

$$Similarity = 1 / \{ Reference value + Penalty value of name ( Japanese kana ) + Penalty value of name ( alphabet ) \}$$

$$= 1 / ( 1 + 1 + 1 )$$

$$= 0.33$$

As the name identification search, the controller 131 calculates the similarity between the search target patient and another patient as described above. The controller 131 extracts a patient having similarity equal to or more than a predetermined value as a related patient. In this manner, the controller 131 functions as a determination section that determines the presence or absence of a related patient satisfying the name identification conditions. The controller 131 also functions as a calculation section that calculates the similarity between the first patient and the related patient. The patient information of the related patient extracted by the name identification search is stored in the RAM 135.

The controller 131 may use items other than the above five items as the name identification conditions. For example, the modality of the imaging apparatus may be included in the name identification conditions. Alternatively, the controller 131 may include the imaging part of the medical image in the name identification conditions. The items to be included in the name identification conditions and the method of determining the presence or absence of a related patient in the name identification search may be appropriately changeable according to a user operation. As described above, the controller 131 functions as a name identification condition setter that sets the name identification conditions for the patient database 21.

Data on the modality of the imaging apparatus and the imaging part of the medical image may be stored in the examination database 22 as the examination information, as described above. Alternatively, the controller 131 may function as an identifying section that identifies such information from the medical images stored in the medical image database 23 using the viewer V or the work list WL.

The controller 131 determines the presence or absence of a related patient in the patient database 21 (step S105). If there is no related patient in the patient database 21 (step S105; No), the controller 131 executes the medical image display processing program P3. Then, as shown in FIG. 5, the controller 131 causes the viewer V to display only the medical image of the search target patient (step 106).

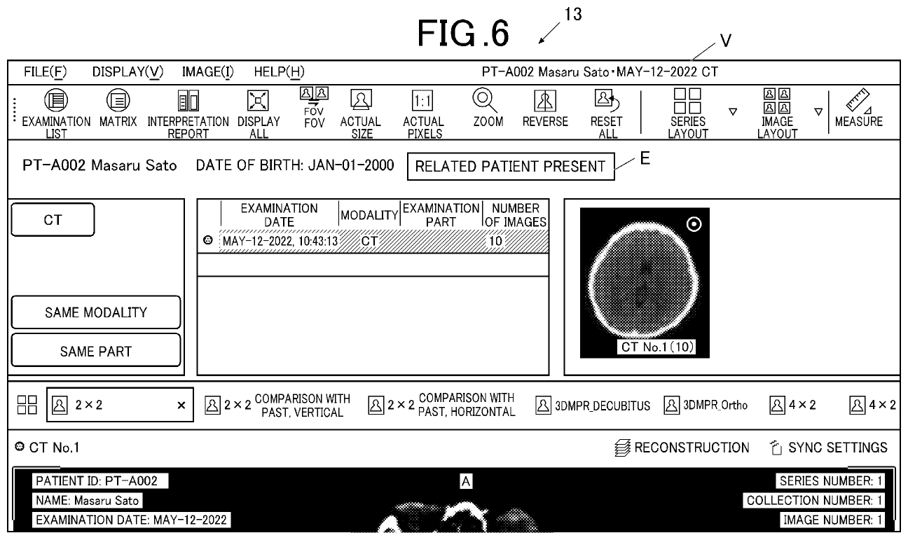

On the other hand, if the associated patient exists in the patient database 21 (step S105; Yes), the controller 131 executes the medical image display processing program P3. As illustrated in FIG. 6, the controller 131 causes the viewer V to display the medical image of the search target patient and existence information E indicating that a related patient exists in the patient database 21 (step S107). Thus, in the present embodiment, the controller 131 functions as a display controller that causes the existence information E to be displayed.

In the present embodiment, the existence information E is, for example, a button including a character, but is not limited thereto. The existence information E may also be, for example, a predetermined symbol or mark.

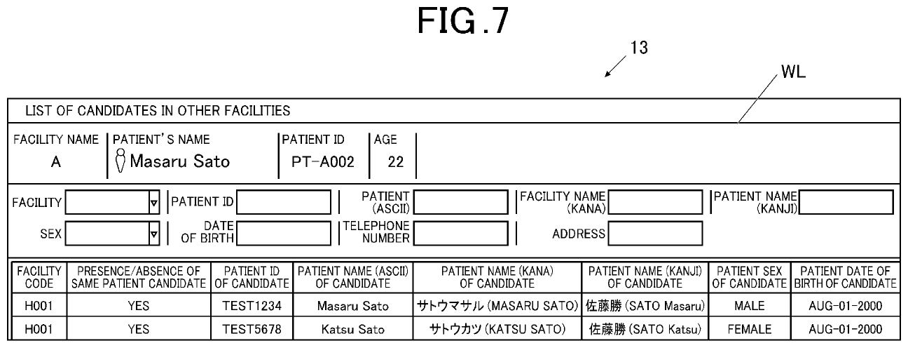

The controller 131 receives a predetermined operation (step S108). In the present embodiment, the predetermined operation is a click on the existence information E. In response to the predetermined operation (step S108; Yes), the controller 131 executes the search result display processing program P2. Next, as illustrated in FIG. 7, the controller 131 displays a list of the patient information of the related patients stored in the RAM 135 in the work list WL (step S109).

The controller 131 receives a predetermined operation on the work list WL (step S110). In the present embodiment, the predetermined operation is a double click for designating one related patient.

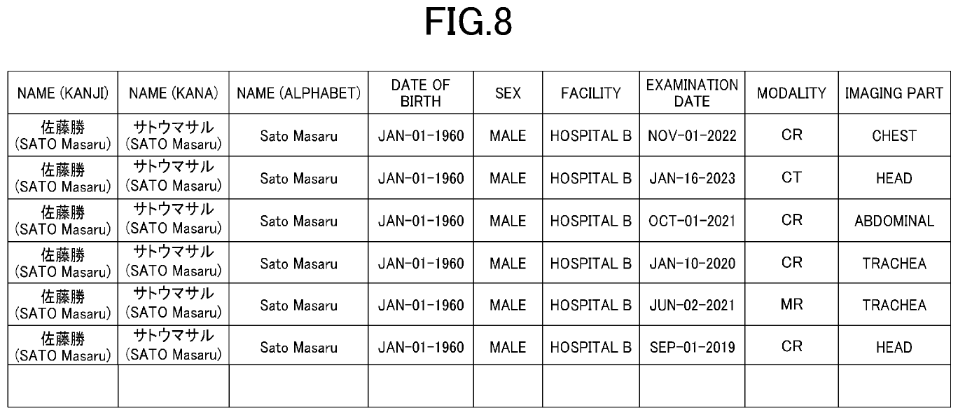

In response to the predetermined operation to the work list WL (step S110; Yes), the controller 131 acquires the examination list for the one related patient from the examination database 22 with the search processing program P1. FIG. 8 shows an example of the examination list. Next, the controller 131 executes the search result display processing program P2 to display the examination list of the specified one related patient in the work list WL (step S111).

The controller 131 receives a predetermined operation on the work list WL (step S112). In the present embodiment, the predetermined operation is, for example, a double click for designating the second examination.

In response to the predetermined operation to the work list WL (step S112; Yes), the controller 131 executes the medical image display processing program P3. Next, the controller 131 displays the medical image of the first examination of the search target patient and the medical image of the second examination of the related patient specified in step S112 together on the viewer V (step S113).

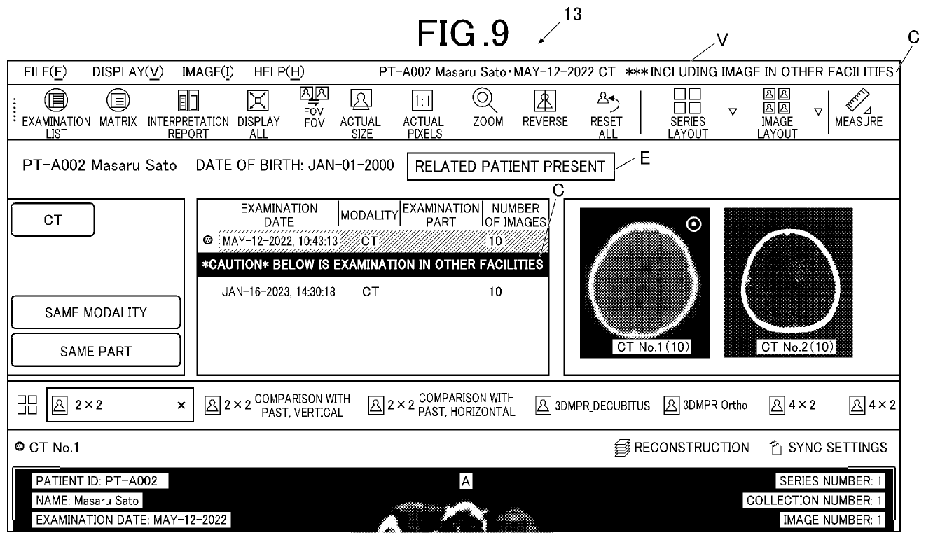

If the examination specified in step S112 is an examination performed in a different medical facility from the examination specified in step S103, it is preferable to display a warning C in step S113. The display is exemplified in FIG. 9. The search target patient and the related patient who have similar person identifying information are patients in different facilities, they may be different patients from each other. Therefore, with the above-described configuration, when the patients are different from each other, the user can readily notice that the patients are different from each other.

### Advantageous Effects of Present Embodiment

As described above, the medical image display apparatus 13 according to the present embodiment includes the display part 133 to display a medical image of the target first patient. A medical image display apparatus 13 includes a communication section 134 for communicating with the patient database 21 for storing person identifying information of patients generated in a plurality of medical facilities. The display part 133 displays the existence information E when the second patient is recorded in the patient database 21. With this configuration, the user can determine whether the necessity of searching for a related patient based on whether the existence information E is displayed on the display part 133. Therefore, it is possible to prevent the search for the related patient in a case where no related patient exists. As a result, it is possible to search for a related patient without reducing the work efficiency of the user.

### [Other Configurations]

Note that the description for the above embodiment illustrates an example of the medical image display apparatus according to the present invention, and the present invention is not limited thereto. The detailed configuration and detailed operation of each section of the apparatus can be appropriately changed without departing from the scope of the present invention.

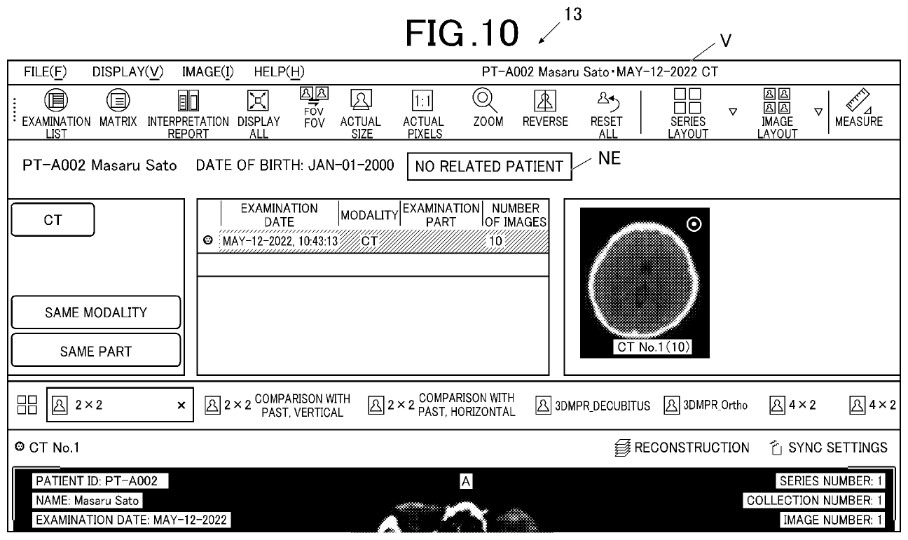

For example, in the above-described embodiment, only the medical image of the search target patient is displayed in a case where no related patient exists. However, the present invention is not limited thereto. For example, as shown in FIG. 10, the controller 131 may display the absence information NE indicating the absence of the related patient on the viewer V. With the above configuration, it is easier to tell the user that a name identification search has been performed but there is no related patient found.

Although the data management apparatus 20 includes the databases corresponding to the respective medical facilities in the above description, it is not limited thereto. That is, a single patient database 21, a single examination database 22, and a single medical image database 23 may centrally manage each data of all the connected medical facilities.

The above description is to disclose an example in which a ROM (appropriately changeable) is used as a computer-readable medium for the program according to the present invention, but the present invention is not limited thereto. As other computer-readable media, a nonvolatile memory such as a flash memory and a portable recording medium such as a CD-ROM are applicable. A carrier wave is also applicable as a medium for providing data of the program according to the present invention via a communication line.

The entire disclosure of Japanese Patent Application No. 2023-048398, filed on Mar. 24, 2023, including description, claims, drawings and abstract is incorporated herein by reference.

What is claimed is:

1. A medical image display apparatus comprising:
a display part that displays a medical image of a target first patient; and
a communicator that communicates with a patient database storing person identifying information on patients generated in a plurality of medical facilities; wherein
the display part displays existence information when a second patient is recorded in the patient database, the second patient being a related patient that has person identifying information similar to person identifying information on the first patient.

2. The medical image display apparatus according to claim 1, wherein
the related patient is a patient presumed to be the first patient.

3. The medical image display apparatus according to claim 1, wherein
the display part displays the existence information before displaying a list of the related patient.

4. The medical image display apparatus according to claim 1, further comprising:
a name identification condition setter that sets a name identification condition for the patient database; and
a determiner that determines presence or absence of a related patient satisfying the name identification condition, wherein
the display part displays the existence information based on a determination result by the determiner.

5. The medical image display apparatus according to claim 4, wherein
the name identification condition includes a modality of an imaging apparatus that was used to capture a medical image of the first patient.

6. The medical image display apparatus according to claim 4, wherein
the name identification condition includes an imaging part of the medical image of the first patient.

7. The medical image display apparatus according to claim 4, wherein
the determiner determines presence or absence of the related patient based on a degree of similarity to the first patient.

8. A medical image display system, comprising:
a medical image display apparatus that displays a medical image of a target first patient and that comprises:
a display part that displays the medical image of the first patient; and
a communicator that communicates with a patient database storing person identifying information on patients generated in a plurality of medical facilities; and
a patient database, wherein
the display part displays existence information when a related patient having person identifying information similar to person identifying information of the first patient is recorded in the patient database.

9. The medical image display system according to claim 8, further comprising:
a specifier that specifies a modality of an imaging apparatus that was used to capture the medical image of the first patient, wherein
the display part displays the existence information when the modality specified by the first specifier is associated with the related patient.

10. The medical image display system according to claim 8, further comprising:
a specifier that specifies an imaging part of the medical image of the first patient, wherein the display part displays existence information when the imaging part specified by the second specifier is associated with the related patient.

11. The medical image display system according to claim 8, further comprising:
a calculator that calculates a similarity between the first patient and the related patient, wherein the display part displays the existence information based on the similarity.

12. A medical image display method, comprising:
displaying a medical image of a target first patient; and
communicating with a patient database storing person identifying information of patients generated in a plurality of medical facilities, wherein
the displaying includes displaying existence information when a related patient having person identifying information similar to person identifying information of the first patient is recorded in the patient database.

13. A computer-readable recording medium storing a program that causes a computer of a medical image display apparatus to function as:
a display controller that causes a display part to display a medical image of a target first patient; and
a communicator that communicates with a patient database storing person identifying information on patients generated in a plurality of medical facilities, wherein
the display controller causes the display part to display existence information when a related patient having person identifying information similar to person identifying information of the first patient is recorded in the patient database.

14. The computer-readable recording medium according to claim 13, wherein
the related patient is a patient presumed to be the first patient.

15. The computer-readable recording medium according to claim 13, wherein
the display controller causes the display part to display the existence information before displaying a list of all of the related patient.

16. The computer-readable recording medium according to claim 13, wherein
the program further causes the computer to function as:
a name identification condition setter that sets a name identification condition for the patient database; and
a determiner that determines presence or absence of the related patient satisfying the name identification condition, and
the display controller causes the display part to display the existence information based on a determination result by the determiner.

17. The computer-readable recording medium according to claim 16, wherein
the name identification condition includes a modality of an imaging apparatus that was used to capture the medical image of the first patient.

18. The computer-readable recording medium according to claim 16, wherein
the name identification condition includes an imaging part of the medical image of the first patient.

19. The computer-readable recording medium according to claim 16, wherein
the determiner determines presence or absence of the related patient based on a similarity to the first patient.
